(19) United States
(12) Patent Application Publication
(10) Pub. No.: US 2005/0279359 A1
(43) Pub. Date: Dec. 22, 2005

(54) Oral suction catheter

(76) Inventors: David M. LeBlanc, Sorrento, LA (US); Daphne S. LeBlanc, Sorrento, LA (US)

Correspondence Address: DAVID L. RAY, ATTORNEY AT LAW, 2051 SILVERSIDE DRIVE, SUITE 205, BATON ROUGE, LA, 70808, US

(21) Appl. No.: 10/870,742

(22) Filed: Jun. 17, 2004

(51) Int. Cl. A62B 9/06; A61M 16/00

(52) U.S. Cl. 128/207.14; 128/207.15

(57) ABSTRACT

An oral suction catheter assembly for removing fluids and secretions from the lungs and trachea of a medical patient having no artificial airways, the oral suction catheter assembly including a hand-held hollow wand having a hollow generally cylindrical opening axially centered therein, the wand having a distal end for insertion into the mouth of a patient, an elongated suction catheter tube received in the hollow wand for extension into the trachea of the patient, and a valve connected to the end of the suction catheter tube for connection to a vacuum source for selectively supplying a vacuum to the suction tube.

### BACKGROUND OF THE INVENTION

1. Field of the Invention

The present invention relates to apparatus for removal of fluids from the lungs of medical patients. In particular, the present invention is related to apparatus for placing a suction catheter in the trachea of a medical patient to remove fluids from the lungs.

2. Description of the Related Art

When it is desired to remove fluids from the trachea or lungs of a medical patient who does not have an implanted artificial airway such as an endotracheal tube, it is common practice to use a process commonly referred to as “nasal tracheal suctioning”. Nasal tracheal suctioning requires a health care practitioner to thread a plastic suction catheter through the nasal passage of a patient and then into the trachea and upper portion of the lungs. A vacuum is selectively applied to the suction to remove fluids from the lungs or trachea through the suction catheter.

Nasal tracheal suctioning causes great pain, discomfort, and trauma to the medical patient. Nasal tracheal suctioning sometimes causes breaking of the delicate bones in the nasal passageway, excessive nasal bleeding, and severe nasal/sinus infections.

Apparatus for removal of fluids from the lungs of medical patients is known in the art. Exemplary of the related art are the following: U.S. Pat. Nos. 6,539,942 B2; 5,779,687; 5,713,849; 5,653,231; 5,643,230; 5,490,503; 5,368,017; 5,259,377; 5,215,522; 5,163,941; 5,038,766; 4,342,315; 4,211,234; 4,454,887; 3,834,388; and 3,968,800; and European Patent Specification EP 0 271 620 B1.

### BRIEF SUMMARY OF THE NVENTION

In accordance with the present invention there is provided an oral suction catheter assembly for removing fluids and secretions from the lungs and trachea of a medical patient having no artificial airways, the oral suction catheter assembly including a hand-held hollow wand having a hollow generally cylindrical opening axially centered therein, the wand having a distal end for insertion into the mouth of a patient, an elongated suction catheter tube received in the hollow wand for extension into the trachea of the patient, and a valve connected to the end of the suction catheter tube for connection to a vacuum source for selectively supplying a vacuum to the suction tube.

### BRIEF DESCRIPTION OF THE SEVERAL VIEWS OF THE DRAWINGS

FIG. 1 is a partly cut-away cross-sectional view of the oral suction catheter apparatus of the invention;

FIG. 2 is a partly cut-away view of a medical patient having the apparatus of the invention inserted into the mouth and trachea of the medical patient; and

FIG. 3 is a partly cut-away view of a medical patient having the apparatus of the invention inserted into the mouth and trachea of the medical patient with the suction catheter extended downward to the entrance to the lungs of the medical patient.

### DETAILED DESCRIPTION OF THE INVENTION

Removing fluids and secretions from the trachea and tracheobronchial tree from a medical patient without an artificial airway such as an endotracheal tube is an important part of the care given to medical patients. Fluids and secretions may be life-threatening in some medical patients having respiratory diseases and disabilities. Suction catheters are utilized to remove fluids and secretions from such medical patients having respiratory diseases and disabilities.

When it is desired to remove fluids and other secretions from the trachea or lungs of a medical patient who does not have an implanted artificial airway, it is common practice to use a process commonly referred to as “nasal tracheal suctioning”. Nasal tracheal suctioning requires a health care practitioner to thread a plastic suction catheter through the nasal passage of a patient and then into the trachea and upper portion of the lungs. A vacuum is selectively applied to the suction to remove fluids from the lungs or trachea through the suction catheter.

Nasal tracheal suctioning causes great pain, discomfort, and trauma to the medical patient. Nasal tracheal suctioning sometimes causes breaking of the delicate bones in the nasal passageway, excessive nasal bleeding, and severe nasal/sinus infections.

The present invention enables health care practitioners to deep suction a medical patient who does not have an artificial airway orally instead of nasally, thereby eliminating most, if not all, of the traumatic side effects of nasal tracheal suctioning. The apparatus of the invention can be introduced orally into the medical patient, and then extended into the trachea for deep suctioning, totally bypassing the nasal sinus areas and avoiding the problems associated with nasal tracheal suctioning. Utilization of the present invention greatly reduces the traumatic side effects of nasal tracheal suctioning, and patient comfort during the suctioning procedure is greatly increased. Comfort of the medical patient is an important goal of the health care practitioner, second only to improving the health of the medical patient to enable the medical patient to be discharge from a health care facility.

Referring now to the drawings, the oral suction catheter assembly of the present invention is generally indicated by the numeral 10. The oral suction catheter assembly 10 of the invention is intended for a single use.

The oral suction catheter assembly 10 of the invention includes a hollow elongated rigid wand generally indicated by the numeral 12 having a hollow generally cylindrical opening 14 axially centered therein. Wand 12 preferably has a generally cylindrical outer surface. Wand 12 is made from a transparent medical grade plastic or polymeric material. Preferably wand 12 has a curved portion generally indicated by the numeral 12a adjacent to the distal end generally indicated by the numeral 12b to facilitate easy insertion into the mouth and throat of a medical patient 18. Wand 12 also preferably has a straight cylindrical portion generally indicated by the numeral 12e to facilitate grasping by the hand generally indicated by the numeral 16 of medical personnel for insertion into the mouth of a medical patient 18.

A hollow suction catheter tube 20 is slidably located inside of hollow generally cylindrical opening 14 of wand 12. Suction catheter tube 20 is made from a semi-rigid plastic or polymeric material well known in the art for common catheter tube construction such as medical grade transparent polyvinyl chloride having an annular wall of essentially uniform thickness throughout having uniform inside and outside diameters. Suction catheter tube 20 has sufficient strength to avoid buckling, bending, and twisting of catheter tube 20 which would occlude or tend to cause occlusion of suction catheter tube 20.

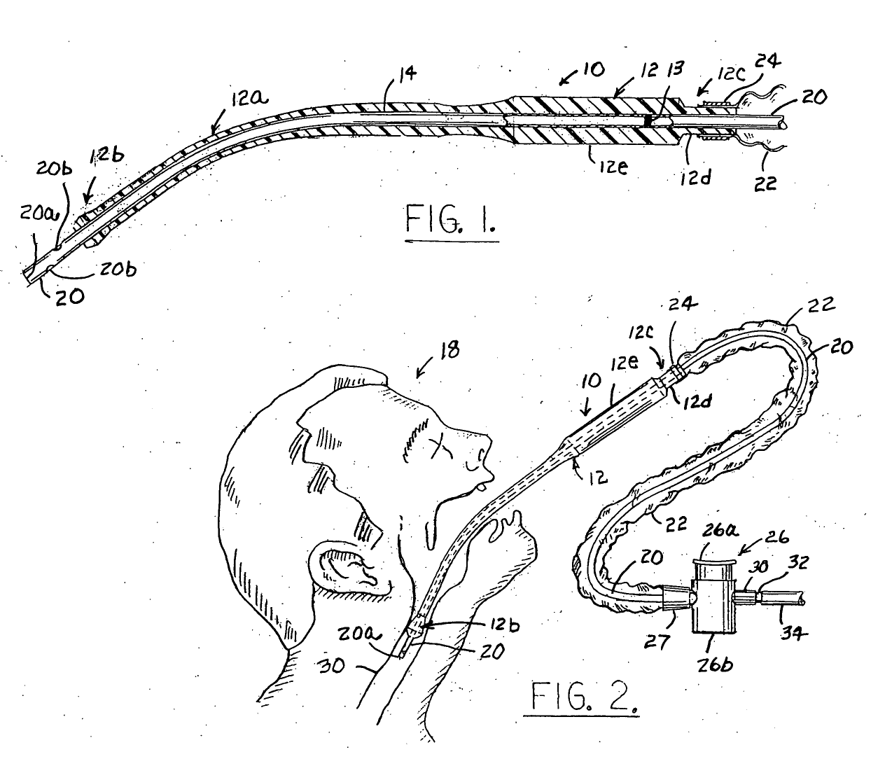

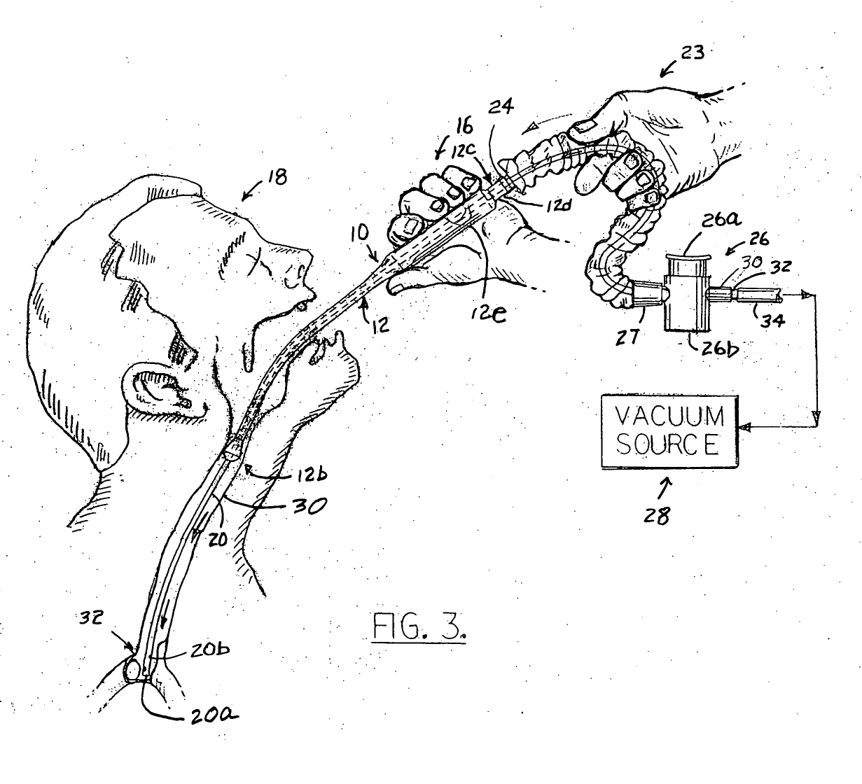

Suction catheter tube 20 is introduced into hollow generally cylindrical opening 14 of the proximal end 12c of wand 12 and can be selectively forced through hollow generally cylindrical opening 14 of wand 12 until the distal end 20a of suction catheter tube 20 extends from the distal end 12b of wand 14 as shown in FIGS. 1-3.

Preferably wand 12 is made from a transparent plastic material to enable suction catheter tube 20 to be viewed inside of wand 12. Preferably suction catheter tube 20 has a black or colored marker 13 shown in FIG. 1 imprinted thereon at a selected distance from the distal end 20a of suction catheter tube 20 which may be viewed through the transparent plastic wand 12 to determine the approximate distance which the distal end 20a of suction catheter tube 20 has been inserted into the trachea of the medical patient 18.

The portion of suction catheter tube 20 extending from the proximal end 12c of hollow elongated rigid wand 12 is encased within and substantially sealed by a transparent tube or sleeve 22 of flexible film of synthetic resinous material, such as medical grade polyethylene. The tubular sleeve 22 prevents contamination of the encircled suction catheter tube 20. Tubular sleeve 22 is manually collapsed, typically between the thumb and index finger of the hand generally indicated by the numeral 23 in FIG. 3 of the attending medical practitioner to grasp and manipulate the suction catheter tube 20 through wand 12 and into the trachea 30 of a medical patient 18.

Hollow elongated rigid wand 12 has a hollow cylindrical nipple 12d at the distal end 12 for receipt of the distal end of tubular sleeve 22. The distal end of tubular sleeve 22 encircles and is firmly connected and sealed to hollow cylindrical nipple 12d by collar 24. Collar 24 may be clamped over tubular sleeve 22 and onto hollow nipple 12 in any conventional manner known in the art.

A normally-closed, manually-operable valve generally indicated by the numeral 26 is firmly connected and sealed to the proximal end of tubular sleeve 22 and the proximal end of suction catheter tube 20 by fastener 27. Valve 26 has a trigger 26a slidable in valve body 26b, which when depressed, causes a suctioning force or vacuum in suction catheter tube 22 to draw or suck fluids in to the distal end 20a of suction catheter tube 20 and holes 20b in suction catheter tube 20 located adjacent to the distal end 20a of suction catheter tube 20. Trigger 26a when depressed into valve body 26b connects the vacuum source labeled in FIG. 3 and generally indicated by the numeral 28 through tubes 30, 32, and 34 to valve 26 and suction catheter tube 20. Vacuum source 28 may be a vacuum pump or a wall mounted vacuum source commonly found in medical patient rooms in health care facilities. Tubular sleeve 22 and valve 26 are known in the art and are disclosed in U.S. Pat. No. 5,215,522, which is hereby incorporated by reference.

As shown in FIG. 3, to utilize the oral suction catheter assembly 10 of the invention to suction or withdraw fluids from a medical patient 18, the straight cylindrical portion 12e of wand 12 is grasped by one hand 16 of a medical practitioner and the distal end 12b of wand 12 is inserted a desired distance into the trachea of a medical patient 18. The other hand 23 of the medical practitioner grasps and collapses tubular sleeve 22, typically between the thumb and index finger of the hand generally indicated by the numeral 23 in FIG. 3 to grasp and manipulate the suction catheter tube 20 through wand 12 and into the trachea 30 of medical patient 18 in the direction indicated by the arrows in FIG. 3 by manual manipulation of suction catheter tube 20 into the trachea 30, tracheobronchial tree 32, or other desired location in the medical patient 18. Trigger 26 is then depressed into valve body 26b to apply a vacuum to suction catheter tube 20 to suck fluids from the medical patient into the distal end 20a of suction catheter tube 20.

Although the preferred embodiments of the invention have been described in detail above, it should be understood that the invention is in no sense limited thereby, and its scope is to be determined by that of the following claims:

1. An oral suction catheter assembly for removing fluids and secretions from the lungs and trachea of a medical patient having no artificial airways, the oral suction catheter assembly comprising:
a. a hand-held hollow elongated transparent rigid wand having a hollow generally cylindrical opening axially centered therein, said hollow elongated transparent rigid wand having a distal end for insertion into the mouth of said medical patient and a proximal end, said hand-held hollow elongated transparent rigid wand having a hand grasping portion located adjacent to said proximal end of said hand-held hollow elongated transparent rigid wand for grasping by one hand of the user of said oral suction catheter assembly to insert said distal end of said hand-held hollow elongated transparent rigid wand into the mouth of said medical patient,
b. an elongated suction catheter tube slidably received in said hollow generally cylindrical opening axially centered in said wand, said suction catheter tube having a distal end for extension from said hollow generally cylindrical opening axially centered in said wand into the trachea of said medical patient, said suction catheter tube having a proximal end extending from said proximal end of said hollow elongated transparent rigid wand, and
c. a valve connected to said proximal end of said elongated suction catheter tube, said valve being for adapted for connection to a vacuum source for selectively supplying a vacuum to said elongated suction catheter tube.

2. The oral suction catheter assembly of claim 1 wherein said hand grasping portion is generally cylindrical in shape.

3. The oral suction catheter assembly of claim 2 wherein said hand-held hollow elongated transparent rigid wand is curved between said distal end of said hand-held hollow elongated transparent rigid wand and said hand grasping portion of hand-held hollow elongated transparent rigid wand.

4. The oral suction catheter assembly of claim 1 wherein said hollow generally cylindrical opening axially centered in said hand-held hollow elongated transparent rigid wand extends from said distal end of hand-held hollow elongated transparent rigid wand to said proximal end of hand-held hollow elongated transparent rigid wand.

5. The oral suction catheter assembly of claim 1 wherein a hollow transparent tubular sleeve is connected to said proximal end of said hand-held hollow elongated transparent rigid wand and said valve for enclosing a portion of said elongated suction catheter tube therein.

6. A method for removing fluids and secretions from the lungs and trachea of a medical patient having no artificial airways, the method comprising:
a. inserting a hand-held hollow elongated transparent rigid wand into the mouth of said medical patient, said hand-held hollow elongated transparent rigid wand having a hollow generally cylindrical opening axially centered therein, said hollow elongated transparent rigid wand having a distal end for insertion into the mouth of said medical patient and a proximal end, said hand-held hollow elongated transparent rigid wand having a hand grasping portion located adjacent to said proximal end of said hand-held hollow elongated transparent rigid wand for grasping by one hand of the user of said oral suction catheter assembly to insert said distal end of said hand-held hollow elongated transparent rigid wand into the mouth of said medical patient,
b. inserting an elongated suction catheter tube into said distal end of said hollow generally cylindrical opening axially centered in said hand-held hollow elongated transparent rigid wand, said elongated suction catheter tube having a distal end and a proximal end,
c. extending said distal end of said elongated suction catheter tube from said proximal end of said hand-held hollow elongated transparent rigid into a selected location in the trachea of said medical patient, and
d. applying a vacuum to said proximal end of said suction catheter tube to draw fluids from said medical patient into said distal end of said elongated suction catheter tube.
